(19) United States
(12) Patent Application Publication
(10) Pub. No.: US 2026/0272124 A1
(43) Pub. Date: Sep. 17, 2026

(54) INSOLE PRESENTATION SYSTEM AND INSOLE PRESENTATION METHOD

(71) Applicant: SK8INSOLL CO., Tokyo (JP)

(72) Inventors: Ryoko Natori, Tokyo (JP); Kazuhiko Kudo, Tokyo (JP); Gary Bruce Beacom, Tokyo (JP)

(73) Assignee: SK8INSOLL CO., Tokyo (JP)

(21) Appl. No.: 19/479,790

(22) PCT Filed: May 1, 2024
(86) PCT No.: PCT/JP2024/016789

§ 371 Date: Oct. 29, 2025

(30) Foreign Application Priority Data
May 3, 2023 (JP) 2023-076380
Feb. 5, 2024 (JP) 2024-016037

(51) Int. Cl. A43D 1/02; A61B 5/103; A63B 24/00

(52) U.S. Cl. CPC A43D 1/02; A61B 5/1038; A63B 24/0075

(57) ABSTRACT

To provide an insole presentation system and an insole presentation method that enable easy acquisition of necessary data with simple measurement and require no expert knowledge related to measurement or proficiency in operating equipment. An insole presentation system includes a foot movement data acquiring unit configured to acquire foot movement data related to the movement of a foot during walking or running, and an insole presenting unit configured to present an insole that is selected based on the foot movement data. The insole is a combination of a base insole that covers an entire rear surface of the foot and a reinforcement insole disposed at a predetermined position of the base insole. The foot movement data includes any one of a degree of plantar flexion/dorsi flexion of an ankle joint, a degree of inversion/eversion of an ankle joint, and a degree of adduction/abduction of an ankle joint.

### TECHNICAL FIELD

The present invention relates to an insole presentation system and an insole presentation method.

### BACKGROUND ART

Conventionally, in a case where shoes are worn while walking or running, insoles are inserted into the soles of shoes for the purpose of improving adhesion between shoes and feet and absorbing shock. Further, it has also been proposed to create an insole suitable for various shapes of feet and walking movement of feet for each individual by acquiring information about the shape of feet and information about the movement of feet at the time of walking (See, for example, Patent Documents 1 to 3).

In a related art shown in Patent Documents 1 to 3, it is possible to reduce the impact applied to a foot during walking and correct the posture for attaining an appropriate walking movement by creating a shape of an insole suitable for each individual in a custom-made or semi-custom-made manner. Thus, it is regarded this prevents injury and illness due to the load applied to the joint and muscles by walking and running.

### RELATED ART DOCUMENTS

### Patent Documents

### [Patent Document 1]

Japanese Patent Publication No. 2007-526028

### [Patent Document 2]

Japanese Patent Publication No. 2017-522917

### [Patent Document 3]

Japanese Laid-Open Patent Publication No. 2022-089563

### SUMMARY OF THE INVENTION

### Problem to be Solved by the Invention

However, the techniques shown in Patent Documents 1 and 2 perform measurement by attaching a dedicated pressure sensor device to acquire the distribution of the load applied to the sole. Such pressure sensor device requires expert knowledge and proficient operation in terms of, for example, attachment methods, measuring methods, and maintenance methods. This results in a problem of making it difficult for users or sellers lacking expertise to properly form an insole.

Further, the technique shown in Patent Document 3 obtains data at various times of movement as comprehensive movement data or five type variant movement data and determines a convex shape of the insole near the hallux in order to correct the movement locus of the COP (Center of Pressure). However, this technique requires a vast amount of data and needs to acquire data by performing multiple complicated movements. This results in a problem of making it difficult to easily create an insole.

Further, Patent Documents 1 to 3 are intended to disperse the load of the sole by way of the shape of the insole. They are not intended to improve the actual walking movement. Further, even if the load of the sole is dispersed by the insole shape, it is difficult to sufficiently improve the walking movement interrelated with complicated elements as well as reduce the load applied to the body.

In view of the above conventional problems, an object of the present invention is to provide an insole presentation system and an insole presentation method which can easily acquire required data with simple measurement and does not require expert knowledge related to measurement nor proficiency in operating a device.

### Means for Solving the Problem

In order to solve the above-described problems, an insole presentation system according to one aspect of the present invention includes: a foot movement data acquiring unit configured to acquire foot movement data related to the movement of a foot during walking or running; and an insole presenting unit configured to present an insole that is selected based on the foot movement data, wherein the insole is a combination of a base insole that covers an entire rear surface of the foot and a reinforcement insole disposed at a predetermined position of the base insole, and wherein the foot movement data includes any one of a degree of plantar flexion/dorsi flexion of an ankle joint, a degree of inversion/eversion of an ankle joint, and a degree of adduction/abduction of an ankle joint.

With such an insole presentation system according to an aspect of the present invention, an insole, which is a combination of a base insole and a reinforcement insole, is selected based on the foot movement data. Thereby, necessary data can be acquired by simple measurement. Further, expert knowledge related to measurement and proficiency for operating equipment would not be required.

Further, according to one aspect of the present invention, the foot movement data includes any one of a degree of plantar flexion/dorsi flexion of an ankle joint, a degree of inversion/eversion of an ankle joint, and a degree of adduction/abduction of an ankle joint.

Further, according to one aspect of the present invention, there is provided a recommended exercise presenting unit that presents recommended exercise information associated with the insole.

Further, according to one aspect of the present invention, the recommended exercise information includes any one of a fascia release method, a walking improvement method, and a training method.

Further, according to one aspect of the present invention, the foot movement data includes any one of a stride length, a foot locus, a foot lifting height, a foot swing speed, and a distance between both feet in a width direction.

Further, according to one aspect of the present invention, there is provided an attribute information acquiring unit that acquires attribute information related to the user of the insole, and the insole presenting unit that presents the insole based on the attribute information.

Further, according to one aspect of the present invention, there is provided a shoe presenting unit that presents shoe information associated with the insole.

Further, according to one aspect of the present invention, there is provided a footprint data acquiring unit that acquires footprint data of a user of the insole, and the insole presenting unit presents the insole based on the footprint data.

In order to solve the above-described problems, an insole presentation system according to one aspect of the present invention includes a foot movement data acquiring unit configured to acquire foot movement data related to the movement of a foot during walking or running; an insole presenting unit configured to present an insole that is selected based on the foot movement data; and a recommended exercise presenting unit configured to present recommended exercise information associated with the insole; wherein the insole is a combination of a base insole that covers an entire rear surface of the foot and a reinforcement insole disposed at a predetermined position of the base insole.

In order to solve the above-described problems, an insole presentation method according to one aspect of the present invention includes a foot movement data acquisition step for acquiring foot movement data related to the movement of a foot during walking or running; and an insole presentation step for presenting an insole selected based on the foot movement data, wherein the insole is a combination of a base insole that covers an entire rear surface of the foot and a reinforcement insole that is disposed at a predetermined position of the base insole, and wherein the foot movement data includes any one of a degree of plantar flexion/dorsi flexion of an ankle joint, a degree of inversion/eversion of an ankle joint, and a degree of adduction/abduction of an ankle joint.

In order to solve the above-described problems, an insole presentation method according to one aspect of the present invention includes a foot movement data acquisition step for acquiring foot movement data related to the movement of a foot during walking or running; an insole presentation step for presenting an insole selected based on the foot movement data; and a recommended exercise presentation step for presenting recommended exercise information associated with the insole.

### Effects of the Invention

According to an embodiment of the present invention, there can be provided an insole presentation system that enables easy acquisition of necessary data with simple measurement and requires no expert knowledge related to measurement or proficiency in operating equipment.

### BRIEF DESCRIPTION OF THE DRAWINGS

FIG. 1 is a schematic diagram showing an outline of an insole presentation system 1000 according to the first embodiment.

FIG. 2 is diagram showing an example of a database of an insole presentation system 1000 in which FIG. 2 (a) illustrates an example of an insole database 340 and FIG. 2 (b) illustrates an example of a recommended exercise database 350. FIG. 3 is a schematic diagram showing an example of an insole presented in an insole presentation system 1000 in which FIG. 3 (a) shows a base insole 10 and FIG. 3 (b) shows reinforcement insoles 11 to 17.

FIG. 4 is a schematic diagram showing the degree of plantar flexion/dorsi flexion of an ankle joint included in the foot movement data in which FIG. 4 (a) illustrates an angle at the time of landing a foot and FIG. 4 (b) illustrates an angle at the time of pushing off a foot.

FIG. 5 is a schematic diagram showing the locus of a foot included in foot movement data.

FIG. 6 is a schematic diagram showing the orientation of a foot included in foot movement data in which FIG. 6 (a) illustrates pronation, FIG. 6 (b) illustrates supination, FIG. 6 (c) illustrates abduction, and FIG. 6 (d) illustrates adduction.

FIG. 7 is a flowchart showing an example of an operation with an insole presentation method using an insole presentation system 1000.

### MODE FOR CARRYING OUT THE INVENTION

### First Embodiment

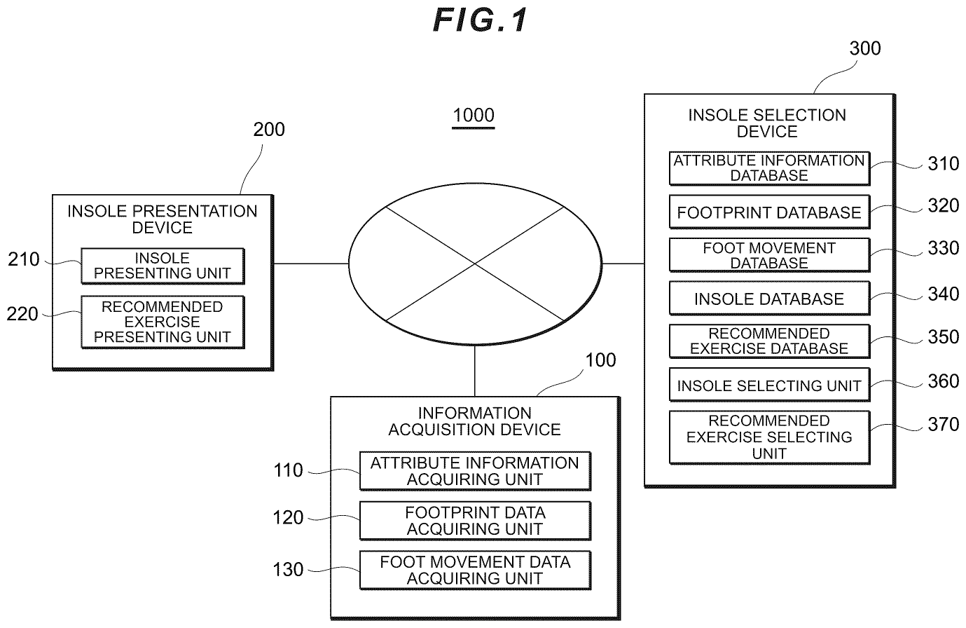

Hereinafter, embodiments of the present invention are described in detail with reference to the drawings. Identical or equivalent components, members, and processes shown in the drawings are denoted by the same reference numerals. Redundant descriptions are omitted as appropriate. FIG. 1 is a schematic diagram showing an outline of an insole presentation system 1000 according to an embodiment of the present invention. As shown in FIG. 1, the insole presentation system 1000 includes an information acquisition device 100, an insole presentation device 200, and an insole selection device 300. Each of the devices performs various functions by executing a predetermined program on a computer provided with, for example, a storage unit, an input unit, an output unit, an arithmetic unit, and an information communication unit. Each of the devices can perform information communication with each other. FIG. 1 illustrates an example in which each of the devices have separate hardware configurations and communicate information with each other through a telecommunication line. Alternatively, multiple programs may be operated by the same hardware configuration to function as each of the devices. Further, multiple functions may be realized on hardware having different configurations and cooperate with each other to function as each of the devices.

The information acquisition device 100 is a device that acquires information necessary for selecting an insole and transmits the information to the insole selection device 300. In the example shown in FIG. 1, the information acquisition device 100 includes an attribute information acquiring unit 110, a footprint data acquiring unit 120, and a foot movement data acquiring unit 130. Various types of information acquired by the information acquisition device 100 are transmitted by an information communication unit to the insole selection device 300 via an electric information communication line or the like. The specific configuration of the information acquisition device 100 is not limited in particular. The information acquisition device 100 may be an information processing device such as a personal computer or a smart phone and may be connected to multiple devices for communicating information with each other.

The attribute information acquiring unit 110 acquires attribute information related to a user of an insole. The specific configuration of the attribute information acquiring part 110 is not limited in particular. The attribute information acquiring unit 110 may be an information processing device such as a personal computer or a smartphone that displays an input form on a screen and receives input by using an input device. Alternatively, information related to a user that is separately recorded may be acquired by an information communication unit. Here, attribute information related to the user may be, for example, age, gender, height, weight, medical history, exercise history, exercise habit, and exercise type.

The footprint data acquiring unit 120 is a part that acquires footprint data that is information related to the shape of a foot of a user of an insole (insole user). The specific configuration of the footprint data acquiring unit 120 is not limited in particular. The footprint data acquiring unit 120 may acquire footprint data by measuring a footprint obtained with plaster or the like and input the footprint data by using an information processing device. Alternatively, a commonly known device dedicated for measuring footprint data may be used. Alternatively, footprint data may be acquired by capturing a stereoscopic image of a foot with use of an imaging device or a LiDAR (Light Detection And Ranging) device mounted on a smartphone or tablet and using an image-recognition technique or a three-dimensional measuring technique. Here, the footprint data includes, for example, the length from the heel to the toe of a foot, the width of a foot, the height of an instep of a foot, the shape of a foot's ground contact surface.

Further, the acquisition of the foot data may be performed by acquiring foot data in a load-applied state in which the foot is contacting the ground or the like or by acquiring foot data in a non-load applied state in which the foot is floated from the ground or the like.

The foot movement data acquiring unit 130 is a part that acquires foot movement data. The foot movement data includes information related to the movement of a foot during a period in which the foot is being moved (foot movement period) when an insole user is walking or running. The specific configuration of the foot movement data acquiring unit 130 is not limited in particular. A method of detecting the locus or the inclination of the foot can be used by inserting an acceleration sensor and a gyro sensor into a shoe that is used during test walking. As an example, a Senno Gait (registered trademark) manufactured by Sennotech Corporation may be used. Further, foot movement data may be acquired by taking a video of the movement of a foot with use of, for example, an imaging device or a LIDAR device mounted on a smartphone or a tablet and using a motion capture technique or an image recognition technique.

Here, the foot movement data may include, for example, the degree of plantar flexion/dorsi flexion of an ankle joint, the degree of inversion (pronation)/eversion (supination) of an ankle joint, the degree of adduction/abduction of an ankle joint, the stride length, the locus of a foot, lifting height of a foot, the swing speed of a foot, and the distance between both feet in the width direction. In this embodiment, the degree of plantar flexion/dorsi flexion of the ankle joint during the foot movement period, the degree of inversion/eversion of the ankle joint, and the degree of adduction/abduction of the ankle joint are important as foot movement data as described below.

The insole presentation device 200 is a device that receives information selected by the insole selection device 300 and presents the information to the user. In the example shown in FIG. 1, the insole presentation device 200 includes an insole presenting unit 210 and a recommended exercise presenting unit 220. The insole presentation device 200 receives the information of the insole selected by the insole selection device 300 and recommended exercise information from the insole selection device 300 by an information communication unit via a telecommunication line or the like and presents the information to the user. The specific configuration of the insole presentation device 200 is not limited in particular. Various information processing devices such as a computer and a smartphone may be used as the insole presentation device 200. Further, commonly known information outputting devices and methods may be used as the method for presenting information such as an image display device, a voice guide device, or a printing device for printing on paper.

The insole presenting unit 210 is a device that receives information of an insole suitable for the user of the insole selected by the insole selection device 300 and presents the information to the user of the insole. Further, the insole presenting unit 210 may include, for example, an online order unit that has a function of accessing an online store connected via a telecommunication line and ordering the presented insole online. Specific details of the insole information presented by the insole presenting unit 210 are described below.

The recommended exercise presenting unit 220 is a device that receives the recommended exercise information associated with the insole selected by the insole selection device 300 and presents the recommended exercise information to the insole user.

Further, the recommended exercise presenting unit 220 may also transfer the recommended exercise information to other devices (e.g., exercise supporting equipment) for use in controlling or adjusting the operation of the exercise supporting equipment.

Although this embodiment shows an example in which the insole presentation device 200 includes the recommended exercise presenting unit 220, the recommended exercise presenting unit 220 may be omitted. Specific details of the recommended exercise information presented by the recommended exercise presenting unit 220 will be described below.

The insole selection device 300 is a device that selects an insole suitable for the insole user based on the information received from the information acquisition device 100 and transmits it to the insole presentation device 200. In the example shown in FIG. 1, the insole selection device 300 includes an attribute information database 310, a footprint database 320, a foot movement database 330, an insole database 340, a recommended exercise database 350, an insole selecting unit 360, and a recommended exercise selecting unit 370. The specific configuration of the insole selection device 300 is not limited in particular. A server device including a storage unit, an information processing unit, and an information communication unit may be used as the insole selection device 300. FIG. 1 shows an example in which the insole selection device 300 is configured in a separate device from the information acquisition device 100 and the insole display device 200. However, the insole selection device 300 may be configured with the same hardware as the information acquisition device 100 and the insole display device 200.

The attribute information database 310 is a part that records attribute information of the insole user acquired by the attribute information acquiring unit 110. The attribute information related to the user recorded in the attribute information database 310 is recorded in association with an identification number assigned to each user. The identification number is also associated with footprint data recorded in the footprint database 320 and with foot movement data recorded in the foot movement database 330.

The footprint database 320 is a part that records footprint data acquired by the attribute information acquiring unit 110. The footprint data recorded in the footprint database 320 is recorded in association with an identification number assigned to each user in the attribute information database 310.

The foot movement database 330 is a part that records foot movement data acquired by the attribute information acquiring unit 110. The foot movement data recorded in the foot movement database 330 is recorded in association with an identification number assigned to each user in the attribute information database 310.

The insole database 340 is a part where information related to the insole selected by the insole selecting unit 360 is recorded. The information related to the insole recorded in the insole selecting unit 360 includes information such as the shape, size, thickness, position, and effect of the base insole 10 and the reinforcement insoles 11 to 17 as described in further detail below.

The recommended exercise database 350 is a part where the recommended exercise information selected by the recommended exercise selecting unit 370 is recorded. The recommended exercise information recorded in the recommended exercise database 350 includes information regarding, for example, a fascia release method, a walking improvement method, a training method, a massage method, and a rehabilitation method as described in further detail below.

The insole selecting unit 360 is a part that selects information of an appropriate insole from the insole database 340 and transmits the information to the insole presentation device 200 based on the foot movement data acquired by the foot movement data acquiring unit 130. Alternatively, attribute information and footprint data associated with the same identification number may be combined for the selection of the insole by the insole selecting unit 360.

The recommended exercise selecting unit 370 is a part that selects appropriate recommended exercise information from the recommended exercise database 350 based on the information of the insole selected by the insole selecting unit 360 and transmits the selected recommended exercise information to the insole presentation device 200. The recommended exercise selection by the recommended exercise selecting unit 370 may be performed by combining attribute information or footprint data associated with the same identification number.

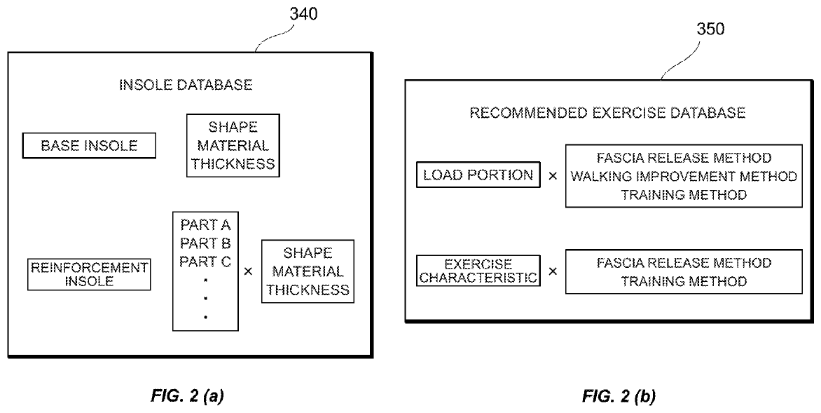

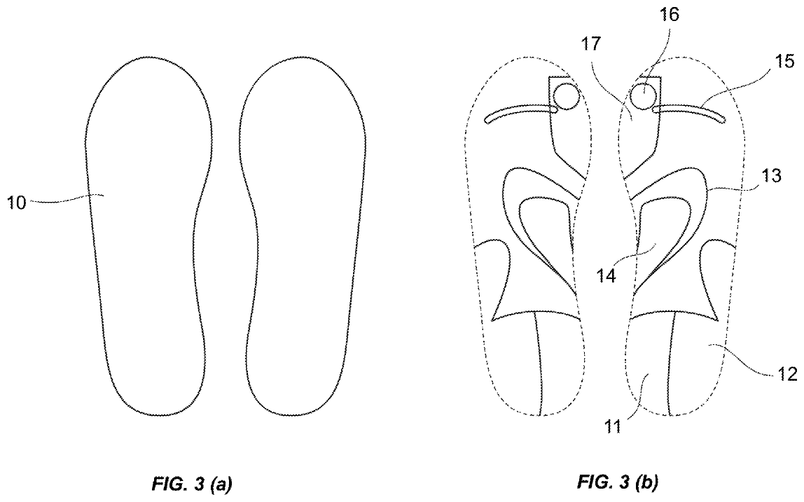

FIG. 2 is a diagram illustrating an example of a database in the insole presentation system 1000 in which FIG. 2(a) illustrates an example of the insole database 340 and FIG. 2(b) illustrates an example of the recommended exercise database 350.

The example shown in FIG. 2 is one example of the insole database 340 and the recommended exercise database 350, each of which may further include a number of data items and subdivided information.

The insole information recorded in the insole database 340 includes information of the base insole 10 covering the entire rear surface of a foot and information of the reinforcement insoles 11 to 17 disposed at predetermined positions of the base insole 10. Further, the information of the base insole 10 includes data items such as the shape, the material, and the thickness of the base insole. Further, the information of the reinforcement insoles 11 to 17 includes multiple parts (e.g. parts A,B,C . . . ) and data items such as the shape, the material, and the thickness of each of the multiple parts. The information of the base insole 10 and the reinforcement insoles 11 to 17 may further include a number of data items or subdivided information such as the size, the characteristics, and the effects of the base insole 10 and the reinforcement insoles 11 to 17.

The recommended exercise information recorded in the recommended exercise database 350 includes multiple pieces of information pertaining to the load area and motion characteristics, respectively. Further, the exercises that are recommended in correspondence with the information of each of the load areas are recorded in association with the information of each of the load areas. Further, the exercises that are recommended in correspondence with the information of each of the exercise characteristics are recorded in association with the information of each of the exercise characteristics. The recommended exercises include, for example, a fascia release method, a walking improvement method, and a training method.

The fascia release method includes, for example, a massaging method or a stretching method for enhancing the extension of fascia at a specific part of a body and the improvement of the range of motion at the specific part of the body. Further, the walking improvement method includes, for example, toe usage, pelvic movement, head position, walking posture, and the center of gravity movement at the sole of a foot. Further, the training method includes, for example, an exercise for training muscular strength of a specific part of a body, a stretching method for expanding the range of motion of a specific part of a body, and a movement practice required according to the type of sport/competition.

FIG. 3 is a schematic diagram showing an example of an insole presented in an insole presentation system 1000 in which FIG. 3 (a) shows a base insole 10 and FIG. 3 (b) shows reinforcement insoles 11 to 17. The base insole 10 has an outer shape that covers the entire rear surface of a foot. The base insole 10 is a part that is inserted into a shoe sole and comes into contact with the sole of the insole user. The reinforcement insoles 11 to 17 are parts to be attached in predetermined positions of the base insole 10 on a rear surface side (ground surface side opposite to the sole of the user's foot) of the base insole 10. The method of attaching the base insole 10 and the reinforcement insoles 11 to 17 is not limited in particular. For example, a method in which the base insole 10 and the reinforcement insoles 11 to 17 are made of a thermoplastic resin and thermally press-bonded may be used. Alternatively, a method of applying an adhesive between the base insole 10 and the reinforcement insoles 11 to 17 and bonding them may be used. By bonding the selected reinforcement insoles 11-17 to the rear surface of the base insole 10, an insole suitable for the insole user can be formed. Although an example of bonding the reinforcement insoles 11 to 17 on the rear surface of the base insole 10 is shown in this embodiment, it may be bonded to the side of the rear surface of the foot of the base insole 10.

As shown in FIG. 3 (a), the base insole 10 has an outer shape covering the entire sole. Here, it is preferred that the entire sole of the foot not only includes a region in which the sole makes contact but also a region of a plantar arch in which the sole does not make contact in a case where the insole is actually inserted into a shoe by the insole user. By providing the base insole 10 to include the region of the plantar arch and the like, the reinforcement insoles 11 to 17 can be satisfactorily bonded and held.

Further, the strength and elasticity of the base insole 10 alone can be ensured.

As shown in FIG. 3(b), the reinforcement insoles 11 to 17 are bonded to predetermined positions of the base insole 10. FIG. 3 (b) shows a state in which all of the reinforcement insoles 11 to 17 are bonded. However, with an actual insole, only those selected from the reinforcement insoles 11 to 17 by the insole selecting unit 360 are bonded.

Although the reinforcement insoles 11 to 17 shown in FIG. 3 (b) are mere examples, a detailed example is described below. The reinforcement insole 11 is positioned and shaped to cover the inside of the calcaneus and the talus. The reinforcement insole 12 is positioned and shaped to cover the outside of the calcaneus and the outside of the cuboid bone. The reinforcement insole 13 is positioned and shaped to cover a wide area of the plantar arch. The reinforcement insole 14 is positioned and shaped to cover a narrow area of the plantar arch. The reinforcement insole 15 is positioned and shaped to extend along from the hallux to the middle phalanx of the toe. The reinforcement insole 16 is positioned and shaped to cover the thenar. The reinforcement insole 17 is positioned and shaped to cover a sesamoid bone of the hallux, a proximal phalanx, a middle phalanx, and a distal phalanx. The shapes and positions of the reinforcement insoles 11 to 17 shown in FIG. 3 (b) are mere examples and may further include a greater number of parts and may be combined into a single part by connecting the parts.

As shown in FIG. 3 (b), the reinforcement insoles 11 to 17 are bonded to predetermined positions of the base insole 10. Alternatively, the reinforcement insoles 11 to 17 may be bonded in positions overlapping each other to form, for example, a combination of the reinforcement insoles 13 and 14 or a combination of the reinforcement insoles 15, 16 and 17. Further, parts of the reinforcement insoles 11 to 17 may be formed overlapping each other. By overlapping the reinforcement insoles 11 to 17, a desired thickness and strength can be easily realized.

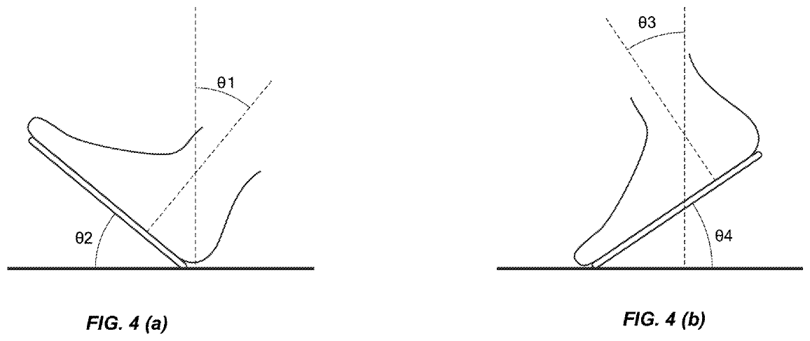

FIG. 4 is a schematic diagram showing the degree of plantar flexion/dorsi flexion of an ankle joint included in the foot movement data in which FIG. 4 (a) illustrates an angle at the time of landing a foot and FIG. 4 (b) illustrates an angle at the time of pushing off a foot. The figure indicates a plane parallel to a ground contact surface with a thick solid line, schematically shows a foot from its tip to the ankle, and illustrates an insole inserted into a shoe sole with a planar outlined shape. Further, the direction perpendicular to the ground and the direction perpendicular to the insole are indicated by broken lines, respectively.

At the time of landing while walking or running, the sole of a foot moving forward touches the ground in sequence in the direction of the toe by landing from the heel of the foot as shown in FIG. 4(a). At this time, the body weight is applied to the sole of the foot, and the load center moves forward in the plane of the sole of the foot. The angle formed between the direction perpendicular to the rear surface of the foot (insole) and the direction perpendicular to the ground contact surface during foot movement period is defined as “θ1”. Further, the angle formed between the rear surface of the foot and the ground contact surface during the foot movement period is defined as “θ2”. In this embodiment, the angles “θ1” and “θ2” between the heel and the ground contact surface in the foot movement period are included in the foot movement data. Accordingly, the foot movement data includes the degree of plantar flexion and dorsi flexion of the ankle joint that can be calculated by the angles “θ1” and “θ2”.

At the time of pushing off of a foot during walking or running, the foot remaining behind separates from the ground contact surface in sequence in a direction from the heel to the toe. At this time, body weight is also applied to the sole of the foot, and the center of load moves forward in the plane of the sole of the foot. The angle formed between the direction perpendicular to the surface of the surface of the sole and the direction perpendicular to the ground contact surface during the foot movement period is defined as “θ3”. Further, the angle formed between the surface of the sole of the foot and the ground contact surface during the foot movement period is defined as “θ4”. In this embodiment, the angles θ3 and θ4 between the toe and the ground contact surface in the foot movement period are included in the foot movement data. Accordingly, the foot movement data includes the degree of plantar flexion and dorsi flexion of an ankle joint that can be calculated by the angles “θ3” and “θ4”.

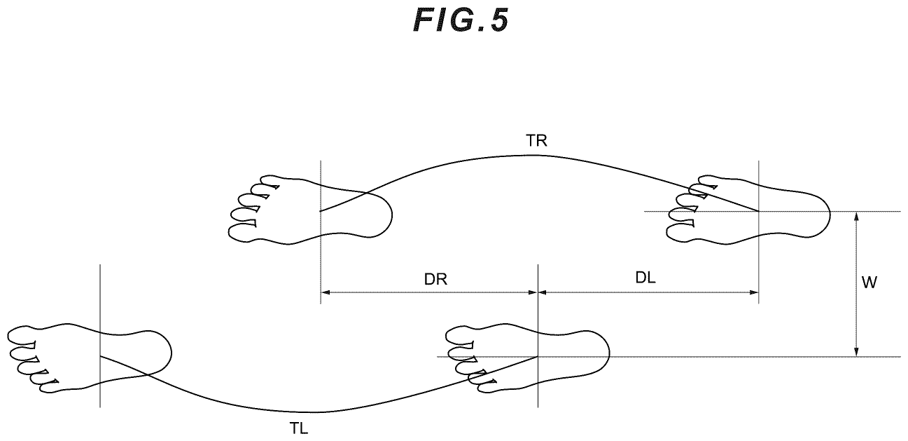

FIG. 5 is a schematic diagram showing the locus of a foot included in foot movement data. The four footmarks shown in the figure indicate the ground contact positions of the right foot and left foot during walking or running by the insole user. Further, the locus of the right foot's movement in the air is shown with a curve TR and illustrated with a solid line, and the locus of the left foot's movement in the air is shown with a curve TL and also illustrated with a solid line. The curves “TR”, “TL”, which are the actual locus of the feet illustrate a three-dimensional locus because they depict the front foot reaching a highest point and then contacting the ground in a process where the rear foot remaining on the ground contact plane is raised and moved forward. Further, “DR” is the distance between ground contact of the left and right feet when the right foot is in front, and “DL” is the distance between the ground contact of the left and right feet when the left foot is in front. Further, “W” is the distance between the ground contacts of the left and right feet in the width direction. In this embodiment, the curves “TR”, “TL”, which are the loci of the feet during walking or running, the ground contact distances “DR, DL” of the left and right feet in the longitudinal direction, and the distance “W” between the ground contacts of the left and right feet in the width direction are included in the foot movement data.

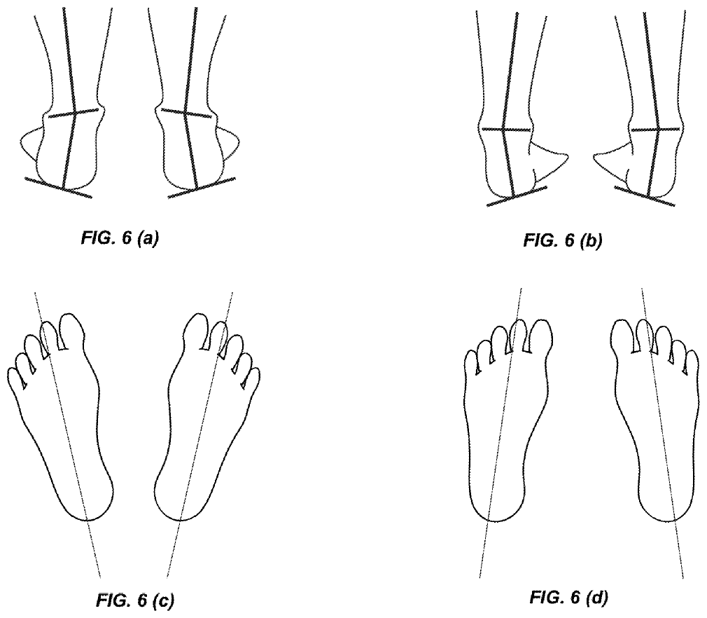

FIG. 6 is a schematic diagram showing the orientation of a foot included in foot movement data in which FIG. 6 (a) illustrates pronation, FIG. 6 (b) illustrates supination, FIG. 6 (c) illustrates abduction, and FIG. 6 (d) illustrates adduction.

FIGS. 6(a) and 6(b) show the view from the heel side of the left and right feet in which the thick solid lines illustrate an ankle joint (talocrural joint), a direction in which a shin bone (tibia) extends, a direction in which a heel bone (calcaneus) extends, and a rear surface of the heel. FIG. 6(a) illustrates a state where the calcaneus extending downward from the talocrural joint opens outward and is pronated (inversion). FIG. 6(b) illustrates a state where the calcaneus extending downward from the talocrural joint closes inward and is supinated (eversion).

FIGS. 6(c) and 6(d) show the directions of the right and left soles in which the straight solid lines indicate the direction extending from the heel to the center of the toenails. FIG. 6(c) illustrates a state where the direction from the heel to the toenails opens outward and exhibits abduction. FIG. 6(d) illustrates a state where the direction from the heel to the toenails closes inward and exhibits adduction. In this embodiment, the foot movement data includes the degree of inversion/eversion of the ankle joint and the degree of adduction/abduction of the ankle joint.

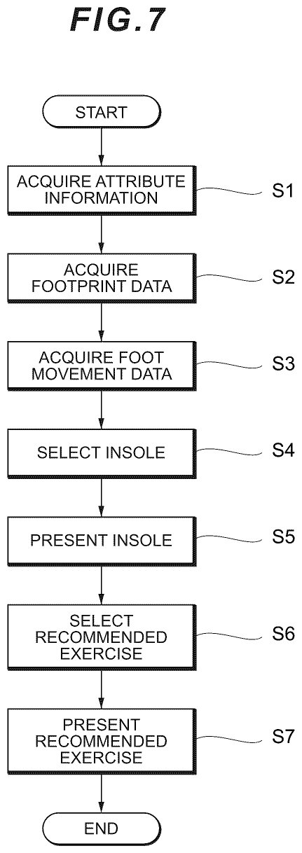

FIG. 7 is a flowchart showing an example of an operation with an insole presentation method using an insole presentation system 1000. The insole presentation method according to this embodiment includes an attribute information acquisition step, a footprint data acquisition step, a foot movement data acquisition step, an insole selection step, an insole presentation step, a recommended exercise selection step, and a recommended exercise presentation step as shown in FIG. 7. In this embodiment, it is assumed that the insole information is recorded in advance in the insole database 340 and the recommended exercise information is recorded in advance in the recommended exercise database 350.

First, in the attribute information acquisition process of Step S1, attribute information of an insole user is acquired by using the attribute information acquiring unit 110. As one example, an insole user inputs attribute information using an input unit such as a mobile information terminal. The attribute information acquiring unit 110 accesses the insole selection device 300 through a telecommunication line or the like and records the acquired attribute information in the attribute information database 310 in association with an identification number. Further, in a case where the insole presentation system 1000 is used by the insole user for the second time or a subsequent time, the attribute information acquiring unit 110 may access the insole selection device 300 through a telecommunication line or the like and read the attribute information from the attribute information database 310. Although this example describes a case where the insole user operates the attribute information acquiring unit 110 by the insole user himself/herself, a clerk or consultant of an insole store may operate the information acquisition device 100.

Next, in the foot data acquisition process of step S2, footprint data of the insole user is acquired by using the footprint data acquiring unit 120. As one example, footprint data, which is measured by an insole user using a tape measure or the like, is input by using an input unit such as a mobile information terminal. The footprint data acquiring unit 120 accesses the insole selection device 300 via a telecommunication line or the like and records the acquired attribute information in association with the identification number in the footprint database 320. Further, in a case where the insole presentation system 1000 is used by the insole user for the second time or a subsequent time, the footprint data acquiring unit 120 may access the insole selection device 300 through a telecommunication line or the like and read the footprint data from the footprint database 320. Although this example describes a case where the insole user inputs the footprint data by himself/herself, a clerk or consultant of the insole store may operate the footprint data acquiring unit 120.

Next, in the foot movement data acquisition process of step S3, foot movement data of the insole user is acquired by using the foot movement data acquiring unit 130. As one example, a measuring device, which incorporates an acceleration sensor, a gyro sensor, and short-range wireless communication or the like, is used. Then, a walking movement or a running movement is performed by the insole user wearing a shoe in a state where the measuring device is placed into the sole of the shoe. The measuring device acquires information such as the orientation of the shoe sole, the movement (travel) distance, and the angle over time and transmits the information to the information acquisition device 100 by short-distance wireless communication or the like. The foot movement data acquiring unit 130 accesses the insole selection device 300 through a telecommunication line or the like and records the acquired foot movement data in the foot movement database 330 in association with an identification number. Measurement by the foot movement data acquiring unit 130 may be performed by the insole user himself/herself or by a clerk or consultant of the insole store.

Next, in the insole selection process of step S4, the insole selecting unit 360 selects an appropriate base insole 10 from the insole database 340 based on the attribute information, the footprint data, and the foot movement data associated with the identification number and further selects an appropriate combination from the reinforcement insoles 11 to 17. As one example, the degree in which the attribute information, the footprint data, and the foot movement data are associated to the base insole 10 and the reinforcement insoles 11 to 17 are recorded as a matrix in the insole selecting unit 360. The insole selecting unit 360 selects the base insole 10 and the reinforcement insoles 11 to 17 having the highest degree of association from the above-described matrix based on the attribute information, the footprint data, and the foot movement data. The insole selecting unit 360 transmits the information related to the selected base insole 10 and the reinforcement insoles 11 to 17 as insole information to the insole presenting unit 210 via a telecommunication line or the like. The insole selecting unit 360 may associate the insole information with the identification number and record the associated information in the attribute information database 310.

Next, in the insole presentation process of step S5, the insole presenting unit 210 receives the insole information from the insole selecting unit 360 and presents the information of the selected insole to the operator of the insole presentation device 200.

The operator of the insole presentation device 200 may be the insole user himself/herself or may be a clerk or consultant of the insole store. As one example, the shape, the material and the thickness of the combination of the selected base insole 10 and the selected reinforcement insoles 11 to 17 are displayed on an image display device. Alternatively, a combination of the selected base insole 10 and the reinforcement insoles 11 to 17 may be displayed as a three-dimensional model on the image display device.
Further, the model number of the selected base insole 10 and the reinforcement insoles 11 to 17 may be displayed instead of displaying the shape, the material, and the thickness of the selected base insole 10 and the reinforcement insoles 11 to 17.

Next, in the recommended exercise selection process of step S6, the recommended exercise selecting unit 370 selects the appropriate recommended exercise information from the recommended exercise database 350 based on the insole information selected by the insole selecting unit 360. Here, the recommended exercise selecting unit 370 may also refer to attribute information, footprint data, and foot movement data associated with the identification number when selecting recommended exercise information. As one example, the recommended exercise selecting unit 370 records the degree of association with the recommended exercise information in a matrix with respect to the base insole 10 and the reinforcement insoles 11 to 17. The recommended exercise selecting unit 370 selects the recommended exercise information having the highest degree of association from the above-described matrix based on the selected insole information. The recommended exercise selecting unit 370 transmits the selected recommended exercise information via a telecommunication line to the recommended exercise presenting unit 220. The recommended exercise selecting unit 370 may associate the recommended exercise information with an identification number and record the information in the attribute information database 310.

Next, in the recommended exercise presentation process of step S7, the recommended exercise presenting unit 220 receives the recommended exercise information from the recommended exercise selecting unit 370 and presents the selected recommended exercise information to the operator of the insole presentation device 200. The operator of the insole presentation device 200 may be the insole user himself/herself or a clerk or consultant of the insole store. As one example, the method of the recommended exercise, the equipment to be used for the recommended exercise, the method of the fascia release, etc. are displayed on the image display device. Further, the actual movement of the recommended exercise may be displayed as a video image. After the recommended exercise presentation process is finished, the insole presentation method according to this embodiment is completed.

As one example of the above-described insole presentation method, the below-described case is where only the right foot is a valgus toe according to the foot shape data, the degree of the plantar flexion/dorsi flexion of the ankle joint is small and the inversion of the ankle joint is exhibited according to the foot movement data. In the insole selection process, the insole is determined by matching the shape of a heel cup of the base insole 10 with the shape of the heel so as to improve the plantar flexion/dorsi flexion of the ankle joint and prevent inversion. Further, the reinforcement insoles 11 and 12 are added to the heel of the left foot because the body weight is applied to the right foot having the valgus toe. Further, the reinforcement insoles 13 and 14 are also added to raise the inner arch for reducing the load on the head of metatarsal bone. The reinforcement insole 16 is added for activating the mechanoreceptor so that the thenar (ball of the thumb) can be firmly pressed and push-off the ground.

Further, in the recommended exercise presentation process, the fascia release of the foot sole, which uses a dedicated oil and a dedicated tool, is proposed because the function of the posterior tibial muscle has deteriorated. Further, a fascia release using a dedicated tool is proposed because the fascia of the entire sole of the foot is stiff. Further, a basic method of using an ankle and a hip joint for effective use of the insole or a method of walking are presented and instructed. By performing these recommended exercises, it becomes easier to perform plantar flexion/dorsi flexion of the ankle joint. Further, it becomes easier for load to be applied to the entire sole of the foot.

As described above, necessary data can be easily acquired by simple measurement because the insole information, which is a combination of the base insole 10 and the reinforcement insoles 11 to 17, is selected based on the foot movement data.

Further, the need for expert knowledge and proficiency of operating equipment can be eliminated.

Further, the above-described foot movement data is preferred to include any one of the degree of plantar flexion/dorsi flexion of an ankle joint, the degree of inversion/eversion of an ankle joint, and the degree of adduction/abduction of an ankle joint.

More preferably, it is preferable to combine the foot movement data with two data items among the degree of plantar flexion/dorsi flexion of an ankle joint, the degree of inversion/eversion of an ankle joint, and the degree of adduction/abduction of an ankle joint. It is further preferable for the foot movement to be combined with all of the aforementioned data items.

The inventor(s) of the present application has found that the selection of the base insole 10 and the reinforcement insoles 11 to 17 based on foot movement data is important, for example, from the viewpoint of reducing the burden applied to the body during walking or running, from the view point of preventing a fall during walking or running, and from the viewpoint of assisting recovery from a state in which walking is difficult to be performed by himself/herself. In order to acquire necessary information for the selection of the insole easily and quickly, it was found that the degree of the plantar flexion/dorsi flexion of an ankle joint, the degree of the inversion/eversion of an ankle joint, and the degree of adduction/abduction of an ankle joint are particularly important among the foot movement data. Further, it was found that the time of the landing of the foot is particularly important during the foot movement period in which foot movement data is measured.

While walking or running, the movement of the foot and the movement of the load as shown in FIG. 5 are repeated, and the load applied to the foot moves from the heel at the timing of landing toward the toe direction. Further, the impact applied to the foot during walking or running is applied first to the heel and becomes highest at the heel. Further, the final point in the locus of the foot during walking or running is at the time of landing of the foot. Further, the angle of the heel at the time of landing differs depending on how the foot is moved. Therefore, the degree of plantar flexion/dorsi flexion of the ankle joint is important among the foot movement data for selecting the optimum insole in order to reduce the burden applied to the body and to promote the appropriate foot movement.

Further, the path when the load moves from the heel to the toe differs depending on the degree of inversion/eversion of the ankle joint at the time of landing of the foot. Further, weight is added from the fibula or tibia to the talocrural joint and calcaneus when the load is moved after landing. Therefore, the degree of inversion/eversion of the ankle joint affects the load applied to, for example, the talocrural joint, the knee joint, and the hip joint. Thus, the degree of inversion/eversion of the ankle joint is also important for selecting the optimum insole to reduce the burden on the body and promote the appropriate foot movement.

Further, the path of the load when the load moves from the heel to the toe differs depending on the degree of adduction/abduction of the ankle joint at the time of landing the foot. Further, when the load moves after landing, the force, that is generated when the ground surface is pushed off by the rear foot, is transmitted from the sole of the foot to the fibula, tibia, talocrural joint, and calcaneus. Therefore, the degree of adduction/abduction of the ankle joint affects the burden applied to, for example, the talocrural joint, the knee joint, and the hip joint. Thus, the degree of adduction/abduction of the ankle joint is also important for selecting the optimum insole to reduce the burden on the body and promote the appropriate foot movement.

Further, since the degree of plantar flexion/dorsi flexion of the ankle joint, the degree of inversion/eversion of the ankle joint, and the degree of adduction/abduction of the ankle joint can be obtained simply by measuring the angle of the ankle, respectively, measurement can be performed easily and quickly. Further, even in a case where the load transfer in the sole cannot be directly measured, the estimation of load transfer or the substitution of load transfer data can be achieved by combining the degree of the plantar flexion/dorsi flexion of the ankle joint, the degree of the inversion/eversion of the ankle joint, and the degree of adduction/abduction of the ankle joint. Thereby, a combination of a suitable base insole 10 and suitable reinforcement insoles 11 to 17 can be easily and quickly selected.

In addition, a more accurate selection of the insole can be achieved by further referring to data including, for example, stride length, foot locus, foot lifting height, foot swing speed, and distance between both feet in the width direction.

Further, a suitable movement can be proposed to the insole user for appropriate walking or running by presenting the recommended exercise information associated with the insole by way of the recommended exercise presenting unit 220. Thus, the combination of the base insole 10 and the reinforcement insoles 11 to 17 not only can assist walking or running but also encourage a walking posture and a walking method that prevent falling or encourage a walking posture and walking method that reduce the burden of joints to the insole user. Thereby, injury and sickness can be prevented.

Further, appropriate rehabilitation information can be provided to, for example, the elderly and those with mobility impairments because the recommended exercise information includes a fascia release method, a walking improvement method, and a training method. This also can provide information useful for prevention of injury and improvement of competitive performance for an athlete performing a specific type of competition.

Further, a more appropriate insole can be selected and presented with the attribute of the insole user taken into consideration owing that attribute information related to the insole user is also acquired by the attribute information acquiring unit 110 and used at the time of selection of the combination of the base insole 10 and the reinforcement insoles 11 to 17 by the insole selecting unit 360.

Further, a more appropriate insole can be selected and presented with the footprint of the insole user taken into consideration owing that footprint data is also acquired by the footprint data acquiring unit 120 and used at the time of selection of the combination of the base insole 10 and the reinforcement insoles 11 to 17 by the insole selecting unit 360.

As described above, necessary data can be easily acquired by simple measurement owing that the insole presentation system 1000 and the insole presentation method according to an embodiment of the present embodiment selects insole information, which is a combination of the base insole 10 and the reinforcement insoles 11 to 17, based on foot movement data. Further, the need for expert knowledge related to measurement and proficiency in operating equipment can be eliminated.

### Second Embodiment

Next, a second embodiment of the present invention is described. Contents overlapping with the first embodiment will not be described. In the first embodiment, the insole selecting unit 360 selects a combination of the base insole 10 and the reinforcement insoles 11 to 17 based on the degree of association with the attribute information, the footprint data, and the foot movement data recorded in the matrix. In this embodiment, selection is performed based on a predetermined relationship.

In one example, foot movement data serving as a target value is recorded in advance in the insole selecting unit 360 as target data. The insole selecting unit 360 calculates the difference between the foot movement data of the insole user and the target data and selects the combination of the base insole 10 and the reinforcement insoles 11 to 17 based on the difference. As a more specific example, in a case where the target data of the degree of the plantar flexion/dorsi flexion of the ankle joint is X degrees and the degree of the plantar flexion/dorsi flexion of the ankle joint acquired by the foot movement data acquiring unit 130 is Y degrees, a combination of the base insole 10 and the reinforcement insoles 11 to 17 is selected based on the difference of the degree of (X−Y). Similarly, the difference between a target data and a measured value may also be calculated and used for insole selection with respect to the degree of inversion/eversion of an ankle joint and the degree of adduction/abduction of an ankle joint.

Further, instead of recording target data in advance, the average value of the measurement results of foot movement data of multiple insole users may be used as target data.

### Third Embodiment

Next, a third embodiment of the present invention is described. Contents overlapping with the first and second embodiments will not be described. In this embodiment, health state history information indicative of the current health state is acquired by the attribute information acquiring unit 110 and recorded in association with an identification number in the attribute information database 310.

Attribute information, footprint data, foot movement data, information of a presented insole, and information of a presented recommended exercise are recorded in association with an identification number in the attribute information database 310.

Accordingly, the use of the insole and recommended exercise and the history of health conditions can be analyzed by recording the health condition history information in the attribute information database 310.

The relationship between the attribute information, the footprint data, the foot movement data, the information of the presented insole, the information of the presented recommended exercise, and the health state history information recorded in the attribute information database 310 may be analyzed by machine learning, so that the insole selecting unit 360 and the recommended exercise selecting unit 370 can refer to the analysis results obtained by the machine learning when selecting a combination of the base insole 10 and the reinforcement insoles 11 to 17 from the next time onward. This further enhances the effect of, for example, preventing injuries and illnesses as well as preventing the decline of physical abilities by utilizing the insoles and recommended exercise.

### Fourth Embodiment

Next, a fourth embodiment of the present invention is described. Contents overlapping with the first to third embodiments will not be described. In this embodiment, the information acquisition device 100 includes a posture data acquiring unit. The posture data acquiring unit acquires posture data which includes data of the posture of the insole user at the time of a standing position or a time of a walking movement by the insole user. The insole selection device 300 includes a posture database. The posture data acquired by the posture data acquiring unit is recorded in association with an identification number in the posture database. Further, a more appropriate insole can be selected and presented with the posture of the insole user taken into consideration owing that the posture data recorded in the posture database is also used at the time of selection of the combination of the base insole 10 and the reinforcement insoles 11 to 17 by the insole selecting unit 360.

### Fifth Embodiment

Next, a fifth embodiment of the present invention is described. Contents overlapping with the first to fourth embodiments will not be described. In this embodiment, the insole selection device 300 includes a shoe database and a shoe selecting unit, and the insole presentation device 200 includes a shoe presenting unit. Further, information related to shoes is recorded in association with the insole in the shoe database. The shoe selecting unit selects appropriate shoe information from the shoe database based on the insole information selected by the insole selecting unit 360, and the shoe presenting unit presents the selected shoe information to an operator of the insole presentation device 200. Further, the insole selecting unit 360 may re-select the shape and material of the insole based on the information of the shoe selected by the shoe selecting unit (e.g., structure and material of an outsole or upper of a shoe).

The present invention is not limited to the above-described embodiments, and various changes can be made within the scope shown in the claims, and embodiments obtained by appropriately combining the technical parts/units disclosed respectively in the different embodiments are also included in the technical scope of the present invention.

### DESCRIPTION OF REFERENCE NUMERALS

1000 . . . Insole presentation system
100 . . . Information acquisition device
200 . . . Insole presentation device
300 . . . Insole selection device
10 . . . Base insole
11-17 . . . Reinforcement insoles
110 . . . Attribute information acquiring unit
120 . . . Footprint data acquiring unit
130 . . . Foot movement data acquiring unit
210 . . . Insole presenting unit
220 . . . Recommended exercise presenting unit
310 . . . Attribute information database
320 . . . Footprint database
330 . . . Foot movement database
340 . . . Insole database
350 . . . Recommended exercise database
360 . . . Insole selecting unit
370 . . . Recommended exercise selecting unit

1. An insole presentation system comprising:
a foot movement data acquiring unit configured to acquire foot movement data related to the movement of a foot in a period in which the foot moves from a time of landing to the next time of landing during walking; and
an insole presenting unit configured to present an insole that is selected based on the foot movement data;
wherein the insole is a combination of a base insole that covers an entire rear surface of the foot and a reinforcement insole disposed at a predetermined position of the base insole, and
wherein the foot movement data includes a degree of plantar flexion/dorsi flexion of an ankle joint, a degree of inversion/eversion of an ankle joint, a degree of adduction/abduction of an ankle joint, and an angle of a heel at the time of landing.

2. The insole presentation system according to claim 1, further comprising:
a recommended exercise presenting unit configured to present recommended exercise information associated with the insole.

3. The insole presentation system according to claim 2, wherein the recommended exercise information includes any one of a muscle release method, a walking improvement method, and a training method.

4. The insole presentation system according to claim 1, wherein the foot movement data includes any one of a stride length, a foot locus, a foot lifting height, a foot swing speed, and a distance between both feet in a width direction.

5. The insole presentation system according to claim 1, further comprising:
an attribute information acquiring unit configured to acquire attribute information related to a user of the insole;
wherein the insole presenting unit is configured to present the insole based on the attribute information.

6. The insole presentation system according to claim 1, further comprising:
a shoe presenting unit configured to present shoe information associated with the insole.

7. The insole presentation system according to claim 1, further comprising:
a footprint data acquiring unit configured to acquire footprint data of the user of the insole;
wherein the insole presenting unit is configured to present the insole based on the footprint data.

8. (canceled)

9. An insole presentation method comprising:
a foot movement data acquisition step for acquiring a foot movement data acquisition step for acquiring foot movement data related to the movement of a foot in a period in which the foot moves from a time of landing to the next time of landing during walking; and
an insole presentation step for presenting an insole selected based on the foot movement data;
wherein the insole is a combination of a base insole that covers an entire rear surface of the foot and a reinforcement insole that is disposed at a predetermined position of the base insole, and
wherein the foot movement data includes a degree of plantar flexion/dorsi flexion of an ankle joint, a degree of inversion/eversion of an ankle joint, a degree of adduction/abduction of an ankle joint, and an angle of a heel at the time of landing.

10. (canceled)

11. The insole presentation system according to claim 2, further comprising:
a footprint data acquiring unit configured to acquire footprint data of the user of the insole;
wherein the insole presenting unit is configured to present the insole based on the footprint data.

12. The insole presentation system according to claim 3, further comprising:
a footprint data acquiring unit configured to acquire footprint data of the user of the insole;
wherein the insole presenting unit is configured to present the insole based on the footprint data.

13. The insole presentation system according to claim 4, further comprising:
a footprint data acquiring unit configured to acquire footprint data of the user of the insole;
wherein the insole presenting unit is configured to present the insole based on the footprint data.

14. The insole presentation system according to claim 5, further comprising:
a footprint data acquiring unit configured to acquire footprint data of the user of the insole;
wherein the insole presenting unit is configured to present the insole based on the footprint data.

15. The insole presentation system according to claim 6, further comprising:
a footprint data acquiring unit configured to acquire footprint data of the user of the insole;
wherein the insole presenting unit is configured to present the insole based on the footprint data.
